(19) United States
(12) Patent Application Publication
(10) Pub. No.: US 2020/0173918 A1
(43) Pub. Date: Jun. 4, 2020

(54) Analysis Method and Analysis Device

(71) Applicant: JVCKENWOOD Corporation, Yokohama-shi (JP)

(72) Inventors: Makoto ITONAGA, Yokohama-shi (JP); Masayuki ONO, Yokohama-shi (JP); Yuichi HASEGAWA, Yokohama-shi (JP); Koji TSUJITA, Yokohama-shi (JP)

(21) Appl. No.: 16/781,258

(22) Filed: Feb. 4, 2020

Related U.S. Application Data
(63) Continuation of application No. PCT/JP2018/029636, filed on Aug. 7, 2018.

(30) Foreign Application Priority Data
Aug. 10, 2017 (JP) 2017-154919

(51) Int. Cl. G01N 21/49; G01N 33/543

(52) U.S. Cl. CPC G01N 21/49; G01N 2201/06113; G01N 33/54366

(57) ABSTRACT

An analysis substrate is irradiated with laser light, and reflected light from reaction region is received to generate a light reception level signal. Particle detection signal having a signal level higher than a predetermined signal level is extracted from the light reception level signal in the reaction region, so as to detect detection target substance in accordance with the extracted particle detection signal. The analysis substrate has the reaction region on which the detection target substance, primary particle provided with antibody for labeling the detection target substance, and secondary particle formed of metal and provided with antigen to be bound to the antibody are captured.

### CROSS REFERENCE TO RELATED APPLICATION

The present application is a continuation of International Application No. PCT/JP2018/029636, filed on Aug. 7, 2018, and based upon and claims the benefit of priority from Japanese Patent Application No. 2017-154919, filed on Aug. 10, 2017, the entire contents of which are incorporated herein by reference.

### BACKGROUND

The present disclosure relates to analysis methods and analysis devices. More particularly, the present disclosure relates to an analysis method and an analysis device for analyzing biomaterials such as antigens and antibodies.

Immunoassays using a sandwich method are known that quantitatively analyze disease detection and therapeutic effects by detecting particular antigens or antibodies as biomarkers associated with diseases.

A method having developed uses an immunoassay to lead antigens to be captured on antibodies fixed on an optical disc, and further modify the antigens with labeling beads, so as to count the number of the antigens as a detection target by an optical method.

JP5958066B discloses a specimen analysis disc for measuring, by optical read-out means, the number of labeling beads bound with biopolymers fixed to track regions on a disc surface having a groove structure or provided with pits.

JP2014-219384A discloses a capturing method including a step of injecting a sample solution including exosomes as a detection target into injection portions having recesses to which antibodies binding to antigens present in the exosomes are fixed, so as to fix the exosomes to the recesses. The capturing method disclosed in JP2014-219384A further includes a modifying step of injecting a buffer solution including beads provided, on the surfaces, with antibodies bound with the antigens included in the exosomes into the injection portions, so as to modify the exosomes with the beads. JP2014-219384A discloses that the number of the beads is counted by laser light emitted from a light source of an optical pickup.

Koji Tsujita et al. (six others), (“Ultrahigh-Sensitivity Biomarker Sensing System Based on the Combination of Optical Disc Technologies and Nanobead Technologies”, Japanese Journal of Applied Physics 52 (2013) 09LB02) discloses a high-sensitivity biomarker sensing system using the combination of optical disc technologies and nanobead technologies. Koji Tsujita et al. discloses that biomarkers as a target are specifically fixed to the surface of an optical disc by an antigen-antibody reaction, and nanobeads are further fixed to the biomarkers. Koji Tsujita et al. discloses that the number of the nanobeads is measured by use of an optical pickup, so as to measure the biomarkers as a target.

### SUMMARY

The conventional methods disclosed in the above documents have a problem described below. During the process of capturing the detection target substances on the analysis substrate by the antigen-antibody reaction, or washing unnecessary unreacted substances, aggregations of protein used for blocking, and salt and a surfactant included in a washing solution may adhere to the analysis substrate as residues.

Residues include various kinds of substances having different sizes or shapes. Detection signals derived from residues of some kind (noise signals) and detection signals derived from particles such as beads (particle detection signals) may have similar pulse waveforms. The conventional analysis methods and the analysis devices cannot distinguish between the noise signals and the particle detection signals having similar pulse waveforms with a high accuracy. When the amount of detection target substances contained is quite small, particles such as beads binding to the detection target substances and captured on the analysis substrate are decreased to a quite small amount. The influence of the noise signals is then relatively increased, leading to a decrease in the accuracy of quantitation of the particles, namely, a decrease in the accuracy (detection limitation) upon the quantitation analysis of the particles including a decrease in detection limitation on the particles or resolution.

In view of the foregoing problems, an object of the present disclosure is to provide an analysis method and an analysis device capable of extracting particle detection signals with a higher accuracy than conventional methods or devices, and detecting detection target substances in accordance with the extracted particle detection signals so as to improve the detection accuracy.

To solve the conventional problem described above, an analysis method according to an aspect of the present disclosure irradiates, with laser light, an analysis substrate formed of resin material and having a reaction region on which a detection target substance, a primary particle provided with an antibody for labeling the detection target substance, and a secondary particle formed of metal and provided with an antigen to be bound to the antibody are captured, receives reflected light from the reaction region to generate a light reception level signal, extracts a particle detection signal having a signal level higher than a predetermined signal level from the light reception level signal in the reaction region, and detects the detection target substance in accordance with the extracted particle detection signal.

To solve the conventional problem described above, an analysis device according to an aspect of the present disclosure includes an optical pickup configured to irradiate, with laser light, an analysis substrate formed of resin material and having a reaction region on which a detection target substance, a primary particle provided with an antibody for labeling the detection target substance, and a secondary particle formed of metal and provided with an antigen to be bound to the antibody are captured, and detect a light reception level of reflected light from the reaction region to generate a light reception level signal, a determination circuit configured to extract a particle detection signal having a signal level higher than a predetermined signal level from the light reception level signal in the reaction region, and a counter circuit configured to detect the detection target substance in accordance with the particle detection signal.

### BRIEF DESCRIPTION OF THE DRAWINGS

FIG. 1 is a top view illustrating an analysis substrate having reaction regions.

FIG. 2 is an enlarged schematic view illustrating a state in which particles are captured on a track region of a reaction region.

FIG. 3 is an enlarged schematic view illustrating a state in which particles specifically binding to detection target substances are captured on the track region of the reaction region.

FIG. 4 is a diagram showing a model used in a simulation.

FIG. 5 is a diagram showing an example of a pulse waveform obtained by the simulation.

FIG. 6 is a table showing a relationship between a complex refractive index of secondary particles and a peak value of a pulse obtained by the simulation.

FIG. 7 is a flowchart illustrating a method of forming the reaction regions on the analysis substrate.

FIG. 8 is a configuration diagram illustrating an analysis device according to the present embodiment.

FIG. 9 is a diagram illustrating a conventional light reception level signal.

FIG. 10 is a diagram illustrating a light reception level signal obtained by an analysis method according to the present embodiment.

FIG. 11 is a flowchart illustrating the analysis method according to the present embodiment.

### DETAILED DESCRIPTION

An analysis method and an analysis device according to the present embodiment are described in detail below. The dimensions of the elements in the drawings may be exaggerated for illustration purposes, and are not necessarily drawn to scale.

[Analysis Device]

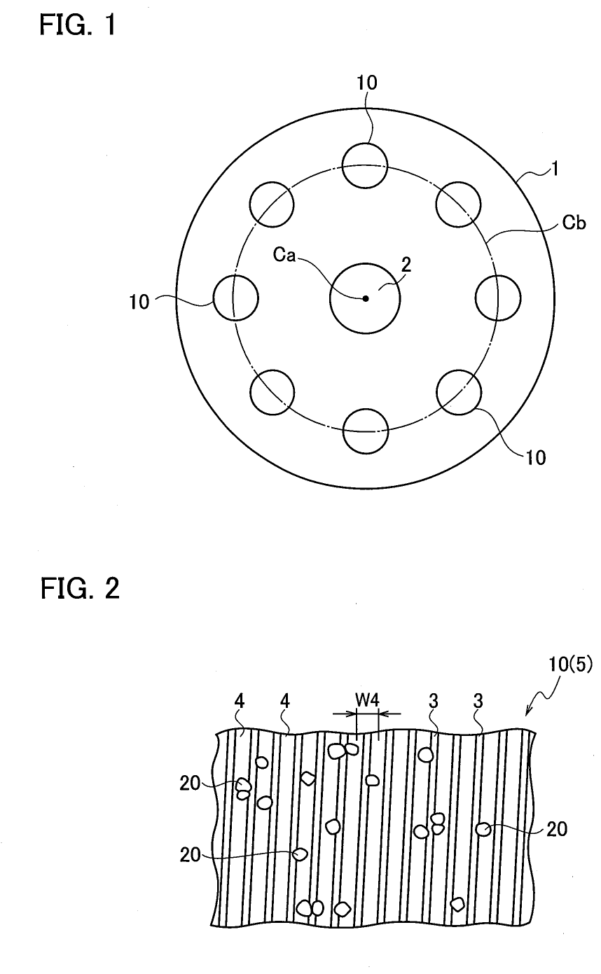

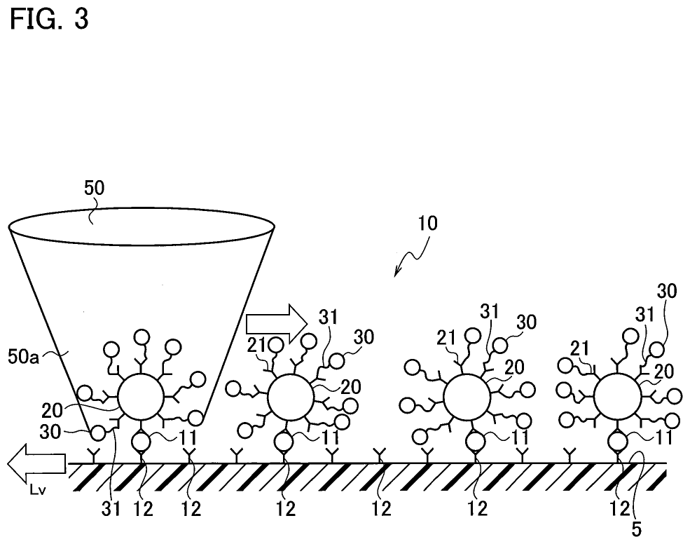

According to the present embodiment, an analysis substrate 1 is used to detect detection target substances 11 (refer to FIG. 3). The analysis substrate 1 used in the present embodiment is described below with reference to FIG. 1 to FIG. 3.

As shown in FIG. 1, the analysis substrate 1 is formed into a circular shape having substantially the same dimensions as optical discs such as Blu-ray discs (BDs), DVDs, and compact discs (CDs).

The analysis substrate 1 is formed of resin material such as polycarbonate resin and cycloolefin polymer, commonly used for optical discs. The analysis substrate 1 is not limited to the optical discs described above, and may be any optical disc having other configurations or conforming to prescribed standards.

The analysis substrate 1 has reaction regions 10. According to the present embodiment shown in FIG. 1, the analysis substrate 1 has a positioning hole 2 in the middle. The eight reaction regions 10 are arranged at regular intervals such that the respective center points are located on the common circle Cb with respect to the center Ca of the analysis substrate 1. The number or the arrangement positions of the reaction regions 10 are not limited to this illustration.

As shown in FIG. 2, the surface of the analysis substrate 1 is provided with track regions 5 having convex portions 3 and recesses 4 alternately arranged in a radial direction. The convex portions 3 and the recesses 4 are formed in a spiral from the inner side to the outer side of the analysis substrate 1. A track pitch W4 of the recesses 4 and the convex portions 3 which is a pitch in the radial direction is 320 nm, for example. The analysis substrate 1 according to the present embodiment is not necessarily provided with the convex portions 3 and the recesses 4, and may be a flat plate.

FIG. 2 and FIG. 3 illustrate the reaction region 10 formed in the track region 5 of the analysis substrate 1. The detection target substances 11, primary particles 20, and secondary particles 30 are captured on the reaction region 10. As shown in FIG. 3, an optical pickup 50 irradiates and scans the reaction region 10 with laser light 50a along the recesses 4, so as to count the number of the detection target substances 11.

The detection target substances 11 are antigens of specific protein associated with a disease, for example. The use of the antigens as the detection target substances 11 can contribute to diagnoses of illnesses, progress observation after treatment, recuperation diagnoses, choice of medicines, companion diagnostics for determining treatment guidelines, and monitoring of illnesses or physical conditions, for example.

The detection target substances 11 such as exosomes vary in concentration in a body fluid depending on the condition of an illness as a target to be monitored, so as to serve as biomarkers. The detection target substances 11 may also be at least one of transmembrane proteins selected from the group consisting of CD9, CD63, CD81, and CEA, which are known as antigens for distinguishing exosomes. When the detection target substances 11 are presumed to be exosomes, an outer diameter of exosomes is typically in a range of 30 nm to 100 nm. When the detection target substances 11 are presumed to be protein, an outer diameter of protein is typically in a range of several to several hundreds of nanometers.

The embodiment shown in FIG. 3 is illustrated with the case in which antibodies 12 specifically bound with the detection target substances 11 are fixed to the regions in which the reaction regions 10 are formed on the track regions 5. The detection target substances 11 are specifically bound to the antibodies 12 fixed to the track regions 5 so that the detection target substances 11 are captured on the track regions 5.

The primary particles 20 are provided with antibodies 21 for recognizing the detection target substances 11. In particular, the plural antibodies 21 specifically bound with the detection target substances 11 are fixed to the surfaces of the primary particles 20. The primary particles 20 specifically bind to the detection target substances 11 captured on the track regions 5 via the antibodies 21. The antibodies 21 of the primary particles 20 specifically bind to the detection target substances 11 so that the primary particles 20 are captured on the track regions 5. The width of the convex portions 3 is preferably set to be less than the width of the recesses 4 so as to lead most of the primary particles 20 to be easily captured on the recesses 4 to improve the detection accuracy if the amount of the detection target substances 11 is small.

The primary particles 20 may be any kind of particles that are provided with the antibodies 21 for recognizing the detection target substances 11, and may be at least one kind of labeling beads selected from the group consisting of polymer particles, metal particles, and silica particles. The primary particles 20 may also be magnetic beads enclosing magnetic material such as ferrite. The use of the magnetic beads can bring the primary particles 20 toward the track regions 5 due to the magnetism, so as to shorten the time necessary for binding the detection target substances 11 with the primary particles 20.

An average particle diameter of the primary particles 20 is preferably, but not necessarily, set in a range of 100 nm to 1 μm. The primary particles 20 with the average particle diameter of 100 nm or greater can facilitate the detection of the detection target substances 11 with a high accuracy. The primary particles 20 with the average particle diameter of 1 μm or smaller can facilitate the counting of the detection target substances 11 with a high accuracy. The average particle diameter of the primary particles 20 is more preferably set in a range of 100 nm to 200 nm. The average particle diameter of the primary particles 20 refers to a particle diameter at 50% of the cumulative value in the particle size distribution in terms of volume, and may be measured by a laser diffraction/scattering method.

The antibodies 21 may be any kind of antibodies that can recognize the detection target substances 11. For example, when the detection target substances 11 are presumed to be exosomes, the antibodies 21 may be at least one kind of antibodies selected from the group consisting of CD9, CD63, CD81, and CEA capable of recognizing antigens. The antibodies 12 and the antibodies 21 may recognize the same antigens or different antigens. When there is one kind of antigens as a target in the detection target substances 11, the antibodies 12 and the antibodies 21 need to recognize different antigens since the primary particles 20 cannot bind to the detection target substances 11 if the antigens to be recognized are the same.

The secondary particles 30 are provided with antigens 31 to be bound to the antibodies 21. In particular, the antigens 31 specifically bound to the antibodies 21 of the primary particles 20 are fixed to the surfaces of the secondary particles 30. The plural secondary particles 30 are specifically bound to the plural antibodies 21 via the antigens 31. The antigens 31 of the secondary particles 30 are specifically bound to the antibodies 21 of the primary particles 20 so that the secondary particles 30 are captured on the track regions 5.

The detection target substances 11, the primary particles 20, and the secondary particles 30 are thus captured on the track regions 5 of the analysis substrate 1. The regions on which the detection target substances 11, the primary particles 20, and the secondary particles 30 are captured are the reaction regions 10 as shown in FIG. 1.

The secondary particles 30 are formed of metal. The secondary particles 30 formed of metal can improve the reflectance of the laser light 50a.

When the secondary particles 30 have a complex refractive index given by n−ki (where n is a refractive index of the secondary particles 30, i is an imaginary unit, and k is an extinction coefficient of the secondary particles 30), the secondary particles 30 preferably fulfill the condition of (k−0.23)²/1.2²+(n−1.36)²/0.94²>1. This relation is led from an optical simulation by a finite-difference time-domain (FDTD) method described below.

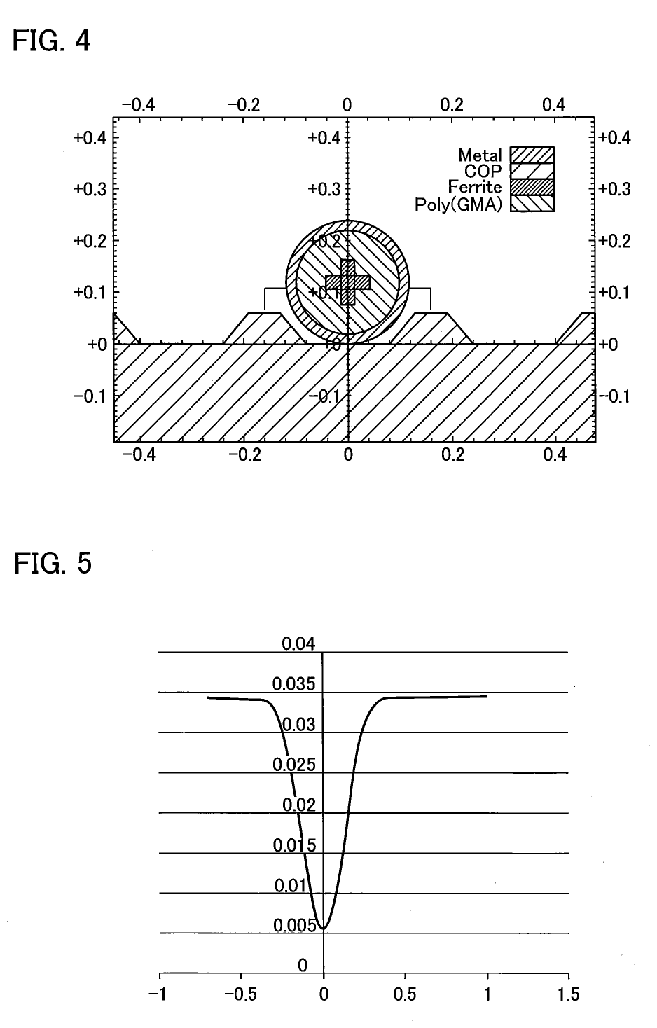

FIG. 4 is a model diagram used in the simulation. The model diagram of FIG. 4 illustrates a state in which a particle is captured on the recess 4 in the analysis substrate 1 formed of cycloolefin polymer (COP). This particle uses a model obtained such that the entire surface of a magnetic bead corresponding to the primary particle 20 is covered with metal. The magnetic bead includes a core portion formed of ferrite, and a base material formed of poly(glycidyl methacrylate) (poly(GMA)) surrounding the core portion so as to be located in the middle of the base material. The covering layer formed of metal covering the surface of the magnetic bead corresponds to the secondary particle 30. This simulation sets the thickness of the covering layer to 20 nm, which is presumed to be an ideal state so as to uniformly cover the entire surface of the primary particle 20. The unit of the values shown in FIG. 4 is a micrometer (μm). For example, the diameter of the magnetic bead composing the primary particle 20 is 200 nm.

FIG. 5 is a diagram illustrating a pulse waveform led by the simulation when n is set to 1.7 and k is set to 0 in the complex refractive index of the secondary particles 30, a wavelength of laser light is set to 405 nm, and the complex refractive index of poly(GMA) is set to 1.53 (n=1.53 and k=0). The axis of abscissa is a position (time), and the axis of ordinate is a reflectance of the laser light. As shown in FIG. 5, the reflectance at the position without particle is about 0.035 (about 3.5%). A peak value of this pulse refers to the reflectance in the middle of the particle, which is 0.006549 (0.6549%).

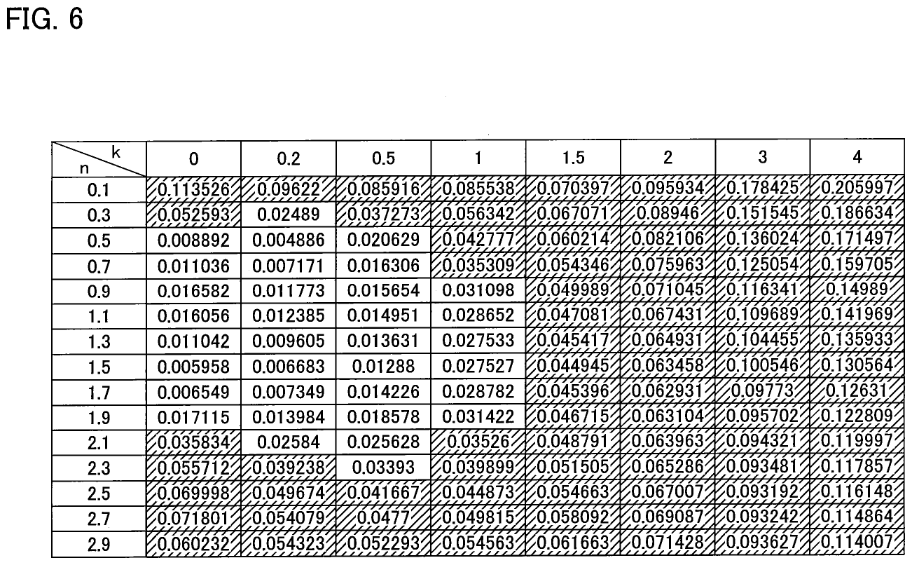

FIG. 6 is a table showing a variation in the peak value of the pulse led by the simulation when the values of n and k in the complex refractive index of the secondary particles 30 are changed. The simulation was carried out under the conditions in which the wavelength of the laser light is set to 405 nm, and the complex refractive index of poly(GMA) is set to 1.53 (n=1.53 and k=0), as in the case described above. The regions without shading in FIG. 6 have the peak value of the pulse which is 0.035 or smaller, while the shaded regions have the peak value of the pulse which exceeds 0.035. The pulse in the regions without shading has a profile of a downward projection, and the pulse in the shaded regions has a profile of an upward projection under the conditions of this simulation.

The boundary between the shaded regions and the regions without shading has a substantially oval profile, and fulfills the condition of (k−0.23)²/1.2²+(n−1.36)²/0.94²=1, according to the calculation. The shaded regions thus fulfil the condition of (k−0.23)²/1.2²+(n−1.36)²/0.94²>1. The present embodiment preferably fulfills the above condition in order to improve the reflectance of the laser light.

According to the results shown in FIG. 6, the complex refractive index of the secondary particles 30 given by n−ki preferably fulfills at least one of the condition of n<0.1 or n>2.5, or the condition of k>1.9 in order to improve the reflectance of the laser light. In other words, it is preferable to fulfill the condition in which n is less than 0.1 or exceeds 2.5, it is preferable to fulfill the condition in which k exceeds 1.9, or it is preferable to fulfill the conditions in which n is less than 0.1 or exceeds 2.5, and k exceeds 1.9. As in the case described above, n is the refractive index of the secondary particles 30, i is the imaginary unit, and k is the extinction coefficient of the secondary particles 30.

The secondary particles 30 are preferably formed of at least one metal selected from the group consisting of gold, silver, platinum, and copper. The secondary particles 30 are more preferably formed of at least one metal selected from the group consisting of gold, silver, and platinum. These metals can further improve the reflectance when the wavelength of the laser light 50a is set to about 405 nm.

An average particle diameter of the secondary particles 30 is preferably, but not necessarily, set in a range of 1 nm to 30 nm. The secondary particles 30 with the average particle diameter of 1 nm or greater can further improve the reflectance of the laser light 50a. The secondary particles 30 with the average particle diameter of 30 nm or smaller lead three-dimensional obstacles to be smaller, so that the primary particles 20 can be covered with a larger number of the secondary particles 30 to further improve the reflectance. The average particle diameter of the secondary particles 30 may be an average value of several to several tens of pieces actually observed with an electron microscope.

The antigens 31 may be any kind of antigens that can be bound to the antibodies 21, but are preferably at least one of protein or fragments of protein. The antigens 31 are preferably fragments of protein in view of purity and availability. The fragments of protein may be peptides including epitopes that can be bound to the antibodies 21, or recombinant protein including epitopes that can be bound to the antibodies 21, for example.

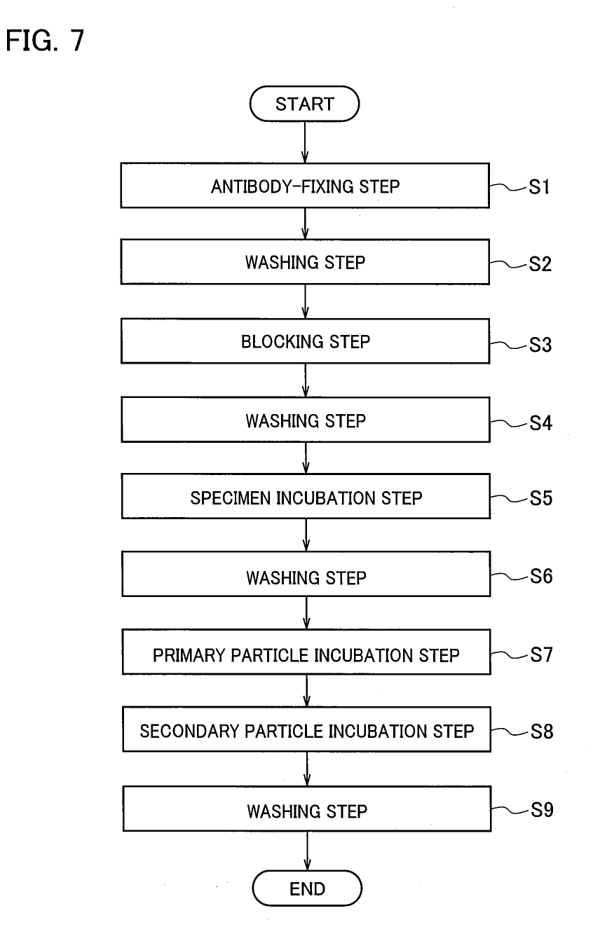

An example of a method of capturing the detection target substances 11, the primary particles 20, and the secondary particles 30 on the reaction regions 10 is described below with reference to FIG. 7. As shown in FIG. 7, the method of forming the reaction regions 10 includes an antibody-fixing step S1, a washing step S2, a blocking step S3, a washing step S4, a specimen incubation step S5, and a washing step S6. The method of forming the reaction regions 10 further includes a primary particle incubation step S7, a secondary particle incubation step S8, and a washing step S9.

In the antibody-fixing step S1, the antibodies 12 specifically bound with the detection target substances 11 as specific antigens associated with a disease are fixed to the regions in which the reaction regions 10 are formed on the track regions 5. For example, a buffer solution including the antibodies 12 is brought into contact with the track regions 5 to be reacted for an appropriate period of time, so as to fix the antibodies 12 to the track regions 5.

In the washing step S2, the track regions 5 are washed after the reacted buffer solution is removed.

In the blocking step S3, the surfaces of the track regions 5 are blocked in order to prevent the antigens from being nonspecifically adsorbed to any portion other than the antigen-recognizing portions of the antibodies 12. In particular, skim milk diluted with a buffer solution is brought into contact with the track regions 5 to be reacted for an appropriate period of time, so as to subject the surfaces of the track regions 5 to blocking treatment. The blocking treatment may use any substance that can achieve similar effects, instead of skim milk.

In the washing step S4, the track regions 5 are washed with a buffer solution after the buffer solution including the skim milk is removed. The buffer solution used for washing may contain skim milk or does not necessarily contain skim milk. The washing step may be omitted.

In the specimen incubation step S5, the detection target substances 11 are specifically bound to the antibodies 12 fixed to the track regions 5. For example, a sample solution including the detection target substances 11 are brought into contact with the track regions 5 to be reacted for an appropriate period of time, so as to lead the detection target substances 11 to be bound to the antibodies 12 by the antigen-antibody reaction and to be captured on the track regions 5.

In the washing step S6, the track regions 5 are washed and dried after the reacted sample solution is removed. The washing step S6 can remove the detection target substances 11 adhering to the surface of the analysis substrate 1 not by the antigen-antibody reaction but by nonspecific adsorption. The sample solution sometimes does not include the detection target substances 11. The embodiment described below is illustrated with the case in which the sample solution includes the detection target substances 11 for illustration purposes.

In the primary particle incubation step S7, the primary particles 20 for labeling the detection target substances 11 are led to specifically bind to the detection target substances 11 captured on the track regions 5. The antibodies 21 specifically bound with the detection target substances 11 are fixe to the surfaces of the primary particles 20. The antibodies 21 of the primary particles 20 specifically bind to the detection target substances 11 so that the primary particles 20 are captured on the track regions 5. The detection target substances 11 and the primary particles 20 are thus captured on the track regions 5 of the analysis substrate 1.

In the secondary particle incubation step S8, the secondary particles 30 for labeling the primary particles 20 are specifically bound to the antibodies 21 fixed to the surfaces of the primary particles 20 captured on the track regions 5. The antigens 31 specifically bound to the antibodies 21 are fixe to the surfaces of the secondary particles 30. The antigens 31 are specifically bound to the antibodies 21 so that the secondary particles 30 are captured on the track regions 5. The detection target substances 11, the primary particles 20, and the secondary particles 30 are thus captured on the track regions 5 of the analysis substrate 1.

In the washing step S9, the track regions 5 are washed and dried after the reacted sample solution is removed.

As described above, the reaction regions 10 which are the regions in which the detection target substances 11, the primary particles 20, and the secondary particles 30 are captured thus can be obtained.

While the embodiment shown in FIG. 7 is illustrated with the case in which the detection target substances 11 are first captured on the track regions 5, and the primary particles 20 are then injected to the track regions 5 so that the primary particles 20 are fixed to the detection target substances 11, the method may inject the detection target substances 11 and the primary particles 20 simultaneously into a buffer solution so as to be reacted with each other. This process has the advantage of a reduction in time necessary for forming the reaction regions 10, since the binding reaction between the detection target substance 11 and the primary particles 20 occurs in the solution.

The embodiment shown in FIG. 7 may change the method of forming the reaction regions 10 as appropriate such that a washing step is added between the primary particle incubation step S7 and the secondary particle incubation step S8, for example.

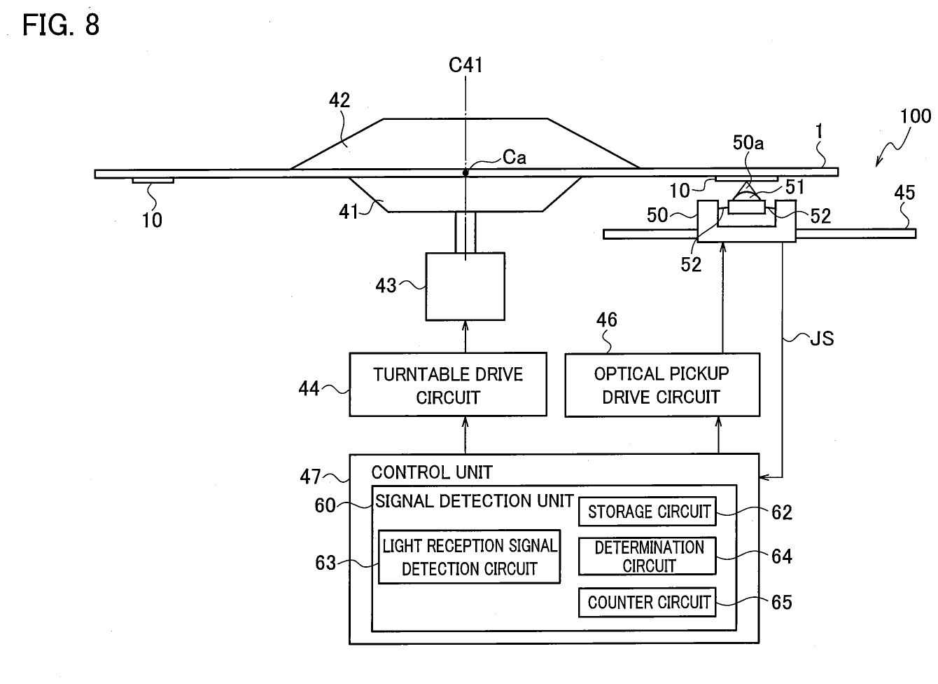

An example of the analysis device according to the present embodiment is described below with reference to FIG. 8. The analysis device 100 according to the present embodiment includes an optical pickup 50, a determination circuit 64, and a counter circuit 65.

As shown in FIG. 8, the analysis device 100 includes a turntable 41, a clamper 42, a turntable drive unit 43, a turntable drive circuit 44, a guide shaft 45, an optical pickup drive circuit 46, a control unit 47, and the optical pickup 50.

The analysis substrate 1 is placed on the turntable 41 with the reaction regions 10 facing down.

The clamper 42 is driven in directions separating from and approaching the turntable 41, namely, in the upper and lower directions in FIG. 8. The analysis substrate 1 is held on the turntable 41 and interposed between the clamper 42 and the turntable 41 when the clamper 42 is driven downward. In particular, the analysis substrate 1 is held such that the center Ca is located on a rotation axis C41 of the turntable 41.

The turntable drive unit 43 drives the turntable 41 to rotate on the rotation axis C41 together with the analysis substrate 1 and the clamper 42. A spindle motor may be used as the turntable drive unit 43.

The turntable drive circuit 44 controls the turntable drive unit 43. For example, the turntable drive circuit 44 controls the turntable drive unit 43 such that the turntable 41 rotates at a constant linear velocity together with the analysis substrate 1 and the clamper 42.

The guide shaft 45 is placed in parallel to the analysis substrate 1 in the radial direction of the analysis substrate 1. The guide shaft 45 is arranged in a direction perpendicular to the rotation axis C41 of the turntable 41.

The optical pickup 50 is supported by the guide shaft 45. The optical pickup 50 is driven along the guide shaft 45 in the radial direction of the analysis substrate 1 and in parallel to the analysis substrate 1. The optical pickup 50 is driven in the direction perpendicular to the rotation axis C41 of the turntable 41.

The optical pickup 50 includes an objective lens 51. The objective lens 51 is supported by suspension wires 52. The objective lens 51 is driven in the directions approaching and separating from the analysis substrate 1, namely, in the upper and lower directions in FIG. 8.

The optical pickup 50 irradiates the analysis substrate 1 with the laser light 50a. The laser light 50a is condensed by the objective lens 51 on the surface of the analysis substrate 1 provided with the reaction regions 10 (on the lower surface of the analysis substrate 1 in FIG. 8). The laser light 50a has a wavelength λ of about 405 nm, for example.

The optical pickup 50 receives the reflected light from the analysis substrate 1. The optical pickup 50 detects a light reception level of the reflected light from the reaction regions 10, and generates a light reception level signal JS. The optical pickup 50 outputs the generated light reception level signal JS to the control unit 47.

The optical pickup drive circuit 46 controls the operation of the optical pickup 50. The optical pickup drive circuit 46 moves the optical pickup 50 along the guide shaft 45 or moves the objective lens 51 of the optical pickup 50 in the vertical direction, for example.

The control unit 47 controls the turntable drive circuit 44 and the optical pickup drive circuit 46. A central processing unit (CPU) may be used as the control unit 47, for example.

The control unit 47 includes a signal detection unit 60 for detecting signals from the analysis substrate 1. The signal detection unit 60 includes a storage circuit 62, a light reception signal detection circuit 63, the determination circuit 64, and the counter circuit 65.

The signal detection unit 60 extracts and counts particle detection signals KS from the light reception level signal JS output from the optical pickup 50, so as to detect and quantitate the detection target substances 11 captured on the reaction regions 10. It is difficult to directly detect the detection target substances 11, since the detection target substances 11 have a size as small as 100 nm. The present embodiment makes use of the high reflectance of the secondary particles 30, so as to indirectly detect and quantitate the detection target substances 11 captured on the reaction regions 10.

The light reception signal detection circuit 63 detects the light reception level signal JS output from the optical pickup 50. In particular, the light reception signal detection circuit 63 detects a pulse wave included in the light reception level signal JS output from the optical pickup 50.

The determination circuit 64 extracts the particle detection signals KS having a signal level higher than a predetermined signal level Lth from the light reception level signal JS in the reaction regions 10. The determination circuit 64 determines, as the particle detection signals KS, the signals in the light reception level signal JS with the signal level higher than the predetermined signal level Lth as a threshold stored in the storage circuit 62.

The predetermined signal level Lth may be set to any level capable of distinguishing between noise signals NS derived from residues and the particle detection signals KS included in the light reception level signal JS. The predetermined signal level Lth is preferably set to a signal level generated when the reflected light is received from a region in which the detection target substances 11 are not present (hereinafter referred to as a “substrate signal level DL”). The reason for this is that the predetermined signal level Lth is determined mainly depending on the condition of the analysis substrate 1, and it is thus easy and accurate to set the predetermined signal level Lth to the substrate signal level DL which is a characteristic value indicating the condition of the analysis substrate 1.

The counter circuit 65 detects the detection target substances 11 in accordance with the particle detection signals KS. In particular, the counter circuit 65 extracts and counts the particle detection signals KS so as to detect and quantitate the detection target substances 11 captured on the reaction regions 10.

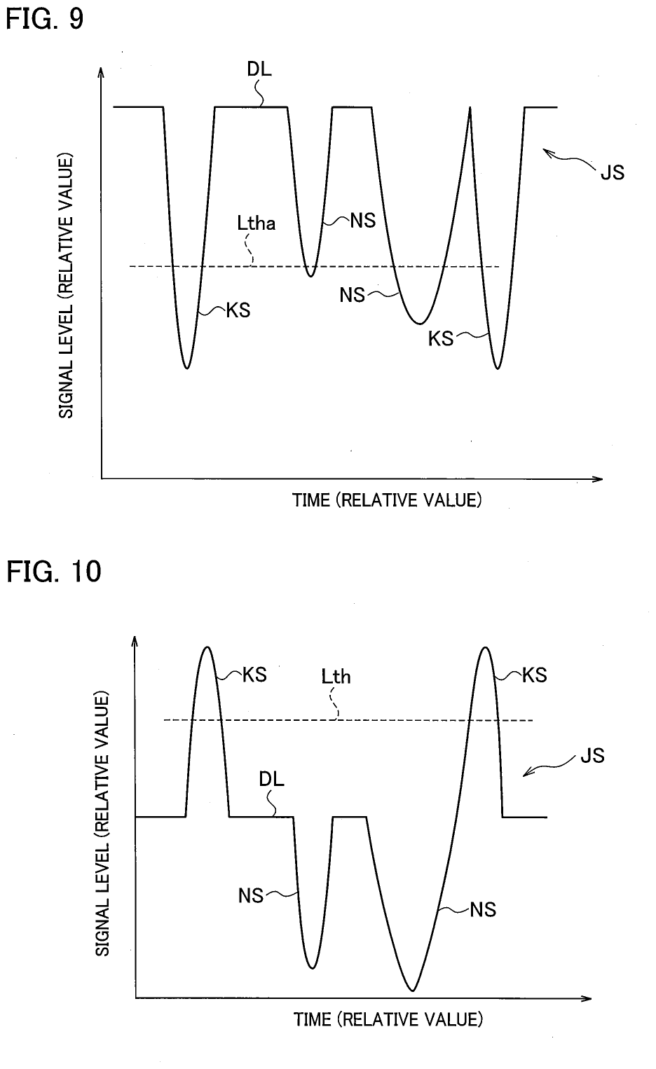

FIG. 9 is a diagram illustrating the light reception level signal JS obtained when typical labeling beads are used. The axis of ordinate in FIG. 9 represents a signal level of the light reception level signal JS, and the axis of abscissa represents a time.

During the formation of the reaction regions 10, aggregations of protein, or salt or a surfactant included in a washing solution may remain as residues in the reaction regions 10. In particular, residues may enter the reaction regions 10 during the step of capturing the detection target substances 11 on the analysis substrate 1 by the antigen-antibody reaction or during the step of washing unnecessary unreacted substances. The noise signals NS derived from such residues may be detected in the light reception level signal JS.

Typical labeling beads are formed of synthesis resin such as polystyrene or epoxy resin. When such resin particles or the residues are irradiated with the laser light 50a, the light tends to be scattered, which decreases the reflectance as compared with the region in which the detection target substances 11 are not present in the analysis substrate 1. When the conventional labeling beads are used for detecting the detection target substances 11, the particle detection signals KS and the noise signals NS are detected in the light reception level signal JS having a signal level lower than the substrate signal level DL, as illustrated in FIG. 9.

The use of the conventional labeling beads could distinguish between the particle detection signals KS and the noise signals NS with some accuracy by comparing the respective signal levels in the light reception level signal JS with a threshold Ltha. However, the conventional labeling beads relatively increase the influence of the noise signals NS if the amount of the detection target substances is quite small. The use of the conventional labeling beads thus cannot improve the accuracy of quantitating the detection target substances, in contrast to the analysis method according to the present embodiment.

The secondary particles 30 according to the present embodiment are formed of metal. The secondary particles 30 thus can increase the reflectance of the laser light 50a, as compared with the case in which the secondary particles 30 are not captured on the reaction regions 10, so as to lead the particle detection signals KS to have a signal level higher than the predetermined signal level Lth, as shown in FIG. 10. FIG. 10 indicates the particle detection signals KS with the signal level (high level) higher than the predetermined signal level Lth in the light reception level signal JS, and the noise signals NS with the signal level (low level) lower than the predetermined signal level Lth in the light reception level signal JS. The substrate signal level DL in the light reception level signal JS is a constant signal level during a period not including either the particle detection signals KS or the noise signals NS.

The present embodiment thus can facilitate the distinction from the noise signals NS with the signal level lower than the predetermined signal level Lth. For example, the light reception level signal JS is compared with the predetermined signal level Lth, so as to accurately extract only the particle detection signals KS from the light reception level signal JS. The primary particles 20 covered with the secondary particles 30 captured on the reaction regions 10 thus can be detected accurately in accordance with the extracted particle detection signals KS.

As described above, the analysis device 100 according to the present embodiment includes the optical pickup 50 configured to irradiate the analysis substrate 1, with the laser light 50a, and detect the light reception level of the reflected light from the reaction regions 10 to generate the light reception level signal JS. The analysis device 100 according to the present embodiment further includes the determination circuit 64 configured to extract the particle detection signals KS having a signal level higher than the predetermined signal level Lth from the light reception level signal JS in the reaction regions 10. The analysis device 100 according to the present embodiment further includes the counter circuit 65 configured to detect the detection target substances 11 in accordance with the particle detection signals KS. The analysis substrate 1 is formed of resin material and has the reaction regions 10 on which the detection target substances 11, the primary particles 20 provided with the antibodies 21 for labeling the detection target substances 11, and the secondary particles 30 formed of metal and provided with the antigens 31 to be bound to the antibodies 21 are captured.

The analysis device 100 according to the present embodiment thus can extract the particle detection signals KS with a higher accuracy than the conventional case, and detect the detection target substances 11 in accordance with the extracted particle detection signals KS, so as to improve the detection accuracy.

[Analysis Method]

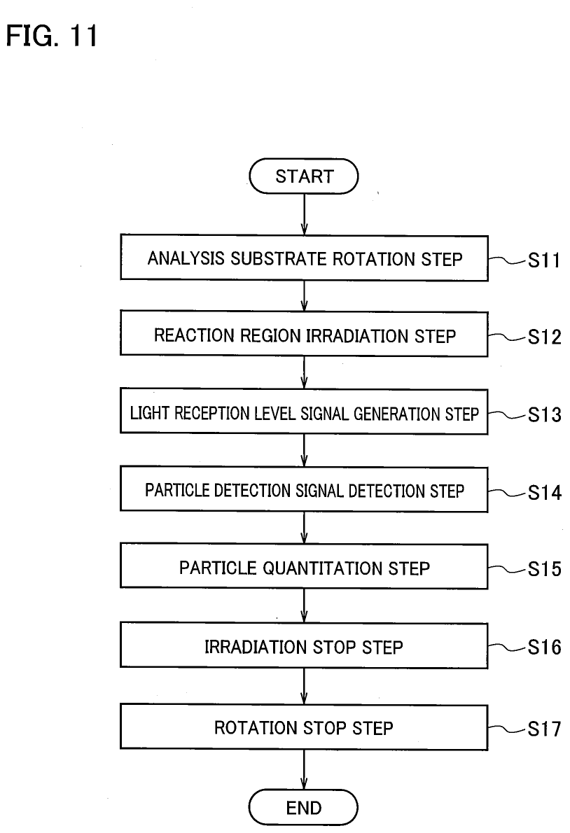

The analysis method according to the present embodiment is described below with reference to the flowchart shown in FIG. 11. Sample solutions sometimes do not include the detection target substances 11. In such a case, the detection target substances 11, the primary particles 20, and the secondary particles 30 are not captured on the reaction regions 10 in the analysis substrate 1. The analysis method is illustrated below with the case in which the detection target substances 11, the primary particles 20, and the secondary particles 30 are captured on the reaction regions 10 for illustration purposes.

An analysis substrate rotation step S11 is a step of rotating the analysis substrate 1. The control unit 47 controls the turntable drive circuit 44 to direct the turntable drive unit 43 to drive the turntable 41 so as to cause the analysis substrate 1 provided with the reaction regions 10 to rotate at the constant linear velocity.

A reaction region irradiation step S12 is a step of irradiating the reaction regions 10 on the analysis substrate 1 with the laser light 50a. The control unit 47 causes the optical pickup 50 to irradiate the analysis substrate 1 with the laser light 50a, and controls the optical pickup drive circuit 46 to move the optical pickup 50 to the radial position at which the reaction regions 10 are formed on the analysis substrate 1. The reaction regions 10 are irradiated with the laser light 50a and are scanned along the recesses 4.

A light reception level signal generation step S13 is a step of receiving the reflected light from the reaction regions 10 to generate the light reception level signal JS. The optical pickup 50 receives the reflected light from the reaction regions 10. The optical pickup 50 detects the light reception level of the reflected light to generate the light reception level signal JS, and outputs the generated light reception level signal JS to the light reception signal detection circuit 63.

A particle detection signal detection step S14 is a step of extracting the particle detection signals KS having a signal level higher than the predetermined signal level Lth from the light reception level signal JS in the reaction regions 10, so as to detect the detection target substances 11 in accordance with the extracted particle detection signals KS. The determination circuit 64 determines, as the particle detection signals KS, the signals in the light reception level signal JS with the signal level higher than the predetermined signal level Lth stored in the storage circuit 62.

The noise signals NS, when included in the light reception level signal JS, have a signal level lower than the substrate signal level DL. The particle detection signals KS with the signal level higher than the predetermined signal level Lth can be easily distinguished from the noise signals NS with the signal level lower than the predetermined signal level Lth. The particle detection signals KS thus can only be extracted from the light reception level signal JS with a high accuracy.

In a particle quantitation step S15, the counter circuit 65 counts the particle detection signals KS, in particular, the number of pulses of the particle detection signals KS for each reaction region 10, and sums up the counted values per track. The detection target substances 11 in the respective reaction regions 10 thus can be quantitated.

In an irradiation stop step S16, the control unit 47 controls the optical pickup drive circuit 46 to move the optical pickup 50 to the initial position, and stops the irradiation of the laser light 50a.

In a rotation stop step S17, the control unit 47 controls the turntable drive circuit 44 to stop the rotation of the turntable 41.

As described above, the analysis method according to the present embodiment irradiates the analysis substrate 1 with the laser light 50a, and receives the reflected light from the reaction regions 10 to generate the light reception level signal JS. The analysis method according to the present embodiment extracts the particle detection signals KS having a signal level higher than the predetermined signal level Lth from the light reception level signal JS in the reaction regions 10, and detects the detection target substances 11 in accordance with the extracted particle detection signals KS. The analysis substrate 1 is formed of resin material and has the reaction regions 10 on which the detection target substances 11, the primary particles 20 provided with the antibodies 21 for labeling the detection target substances 11, and the secondary particles 30 formed of metal and provided with the antigens 31 to be bound to the antibodies 21 are captured.

The analysis method according to the present embodiment thus can extract the particle detection signals KS with a higher accuracy than the conventional case, and detect the detection target substances 11 in accordance with the extracted particle detection signals KS, so as to improve the detection accuracy.

### EXAMPLES

The present embodiment is described in more detail below with reference to an example and a comparative example, which are not intended to limit the present embodiment.

### Example 1

First, antibodies for recognizing CD9 which is antigen protein specific to exosomes were fixed to reaction regions in an optical disc substrate. The optical disc substrate was then washed with a washing solution.

Next, a specimen including exosomes was brought into contact with the reaction regions so that the exosomes in the specimen were captured on the optical disc substrate. The optical disc substrate was then washed with a washing solution.

Next, antibodies for recognizing CEA which is protein specific to the exosomes and presumed to be associated with various kinds of cancer were fixed to the surfaces of silica beads so as to prepare primary particles. The primary particles were brought into contact with the reaction regions to bind to the exosomes captured on the optical disc substrate, so as to lead the primary particles to be captured on the optical disc substrate. The optical disc substrate was then washed with a washing solution.

Next, CEA recombinant protein was fixed to the surfaces of silver nanoparticles so as to prepare secondary particles. The secondary particles were brought into contact with the reaction regions to be bound to the antibodies 21 of the primary particles captured on the optical disc substrate, so as to lead the secondary particles to be captured on the optical disc substrate. The optical disc substrate was then washed with a washing solution, so as to prepare an analysis substrate provided with the exosomes as detection target substances.

### Comparative Example 1

An analysis substrate was prepared in the same manner as Example 1, except that the secondary particles were not captured on the optical disc substrate.

[Evaluation]

Exosomes expressing CD63 tend to be present with a greater amount than exosomes expressing CEA, and could be sufficiently detected by a conventional method. The amount of exosomes expressing CEA is quite small, which is 1% or smaller, as compared with exosomes expressing CD63. The following evaluation was performed so as to determine whether the exosomes with such a quite small amount can be detected. The specific evaluation method is described below.

First, the reaction regions were irradiated with laser light having a wavelength of 405 nm. While a signal level generated when the reflected light was received from a region in which the detection target substances were not present was determined as a predetermined signal level, signals obtained from the reflected light from the reaction regions and having a signal level higher than the predetermined signal level were determined as particle detection signals. The signal level of the particle detection signals was compared with the predetermined signal level, so as to count the number of the exosomes as the detection target substances.

Evaluation results revealed that Example 1 using the secondary particles could clearly distinguish between the signals derived from the exosomes and the signals derived from noise, since the signals derived from the exosomes had a signal level higher than the predetermined signal level.

Comparative Example 1 not using the secondary particles could not make a clear distinction between the signals derived from the exosomes and the signals derived from noise, since the signals derived from the exosomes had a signal level lower than the predetermined signal level.

Since the amount of CEA included in the exosomes is quite small, which is 1% or smaller, as compared with CD63, it is difficult to detect the exosomes only with the primary particles, as illustrated in Comparative Example 1. The use of the secondary particles can improve the reflectance of the laser light, as illustrated in Example 1. The analysis substrate prepared in Example 1 thus can detect the small amount of the detection target substances with a high accuracy.

The amount of exosomes expressing CD63 is relatively large, as described above. When exosomes including protein expressed with a small amount are analyzed as a detection target, the distinction between the noise signals and the particle detection signals may be difficult by a conventional method. The present embodiment described above can detect the detection target substances with a high accuracy regardless of whether the amount of exosomes included in a specimen is quite small.

While the present embodiment has been described above by reference to the examples, the present embodiment is not intended to be limited to the above descriptions, and various modifications and improvements will be apparent to those skilled in the art.

What is claimed is:

1. An analysis method comprising:
irradiating, with laser light by an optical pickup, an analysis substrate having a reaction region on which a detection target substance is bound to the reaction region, the detection target substance is bound to a primary particle provided with an antibody to be bound to the detection target substance, the primary particle is bound to a secondary particle, having a complex refractive index different from the primary particle, formed of metal and provided with an antigen to be bound to the antibody;
receiving reflected light from the reaction region to generate a light reception level signal by the optical pickup;
extracting a particle detection signal having a signal level higher than a predetermined signal level from the light reception level signal in the reaction region by a determination circuit; and
detecting the detection target substance in accordance with the extracted particle detection signal by a counter circuit,
wherein the light reception level signal derived from the primary particle bound to the secondary particle is higher than the predetermined signal level.

2. The analysis method according to claim 1, wherein the predetermined signal level is a signal level generated when reflected light is received from a region in which the detection target substance is not present.

3. The analysis method according to claim 1, wherein the secondary particle having the complex refractive index given by n−ki (where n is a refractive index of the secondary particle, i is an imaginary unit, and k is an extinction coefficient of the secondary particle) fulfills a condition of (k−0.23)²/1.2²+(n−1.36)²/0.94²>1.

4. The analysis method according to claim 1, wherein the secondary particle is formed of at least one metal selected from the group consisting of gold, silver, platinum, and copper.

5. The analysis method according to claim 1, wherein the antigen is at least one of protein or a fragment of protein.

6. An analysis device comprising:
an optical pickup configured to irradiate, with laser light, an analysis substrate having a reaction region on which a detection target substance is bound to the reaction region, the detection target substance is bound to a primary particle provided with an antibody to be bound to the detection target substance, the primary particle is bound to a secondary particle, having a complex refractive index different from the primary particle, formed of metal and provided with an antigen to be bound to the antibody, and the optical pickup is configured to detect a light reception level of reflected light from the reaction region to generate a light reception level signal;
a determination circuit configured to extract a particle detection signal having a signal level higher than a predetermined signal level from the light reception level signal in the reaction region; and
a counter circuit configured to detect the detection target substance in accordance with the particle detection signal,
wherein the light reception level signal derived from the primary particle bound to the secondary particle is higher than the predetermined signal level.
